(19) United States
(12) Patent Application Publication
(10) Pub. No.: US 2011/0300091 A1
(43) Pub. Date: Dec. 8, 2011

(54) TRANSLUCENT DEODORANT STICKS CONTAINING SOLUBILIZED 3,4,4'-TRICHLOROCARBANILIDE

(75) Inventors: Robert Demson, Cave Creek, AZ (US); Robert Dail, Phoenix, AZ (US); Jennifer Harris, Phoenix, AZ (US)

(73) Assignee: The Dial Corporation, Scottsdale, AZ (US)

(21) Appl. No.: 12/793,392

(22) Filed: Jun. 3, 2010

(51) Int. Cl. A61K 8/40; A61K 8/92; A61Q 15/00

(52) U.S. Cl. 424/65

(57) ABSTRACT

The present invention is a solid translucent deodorant stick that exhibits remarkable efficacy against gram positive bacteria. Maintenance of translucency in spite of a high level of antimicrobial is possible through use of solubilized 3,4,4′-trichlorocarbanilide. The solubilized TCC is formed as a premix which is then added to the some or all of the remaining ingredients comprising a stearate-gelled glycol solvent stick.

### PRIORITY APPLICATION

This application claims the benefit of and priority to U.S. Provisional Patent Application No. 61/183,338 filed Jun. 2, 2009 and entitled “TRANSLUCENT DEODORANT STICKS CONTAINING SOLUBILIZED 3,4,4′-TRICHLOROCARBANILIDE”, which is incorporated by reference herein it its entirety.

### FIELD OF INVENTION

The present invention relates to deodorant compositions and more particularly to translucent solid personal care deodorant compositions wherein the active ingredient is solubilized 3,4,4′-trichlorocarbanilide.

### BACKGROUND OF THE INVENTION

Personal care deodorant compositions in solid form have been in the market for decades. Personal care deodorants are typically formulated to work through a combination of fragrance technology and antimicrobial activity. Most notably, a deodorant can be formulated to include a fragrance accord that helps mask the odor associated with human perspiration. Additionally, an antimicrobial active may be incorporated in a deodorant composition to destroy or at least limit odor causing bacteria found on perspiring human skin. The most common antimicrobial active for personal care deodorants has been 2,4,4′-trichloro-2′-hydroxy diphenyl ether (triclosan). Additionally, some glycols such as propylene glycol and dipropylene glycol function as bacteriostats. Triclosan and the glycols have found the most use in underarm deodorants because of their low cost, low irritancy and marked solubility in a host of carriers. For example, triclosan is easily incorporated in stick deodorants having either a stearate gelled glycol solvent base (soap stick) or a wax base (wax stick). However, both glycols and triclosan have the shortcomings that although they exhibit broad spectrum efficacy, they are only weakly such, and do not specifically target gram positive bacteria. Thus, deodorants incorporating only these materials may become ineffective over time as the gram positive bacteria begin to multiply in the human perspiration.

3,4,4′-Trichlorocarbanilide, (also known as N-(4-chlorophenyl)-N′-(3,4-dichlorophenyl)urea; TCC; trichlorocarbanilide; triclorocarban; and, triclocarban) on the other hand has excellent gram positive bactericidal activity, but its solubility in a deodorant composition such as a gelled glycol solvent based composition is terrible. The molecule is a neutral urea, with hydrophobic aryl groups, and is not ionized at near neutral pH. Thus, there is no obvious way to incorporate the insoluble triclocarban in a stearate-gelled glycol solvent stick and still retain translucency. Of course the triclocarban could be dispersed as an insoluble particle throughout the stick, but that could lead to an inhomogeneous deodorant stick, with the particles settling as the stick solidifies in manufacturing. Additionally, the efficacy of the TCC may be reduced since it would only be left on the skin as insoluble particles, unable to absorb into bacteria.

A perusal of the prior art demonstrates a need for translucent solid deodorant compositions that employ the gram positive bacteriocide 3,4,4′-trichlorocarbanilide at bactericidal levels.

### SUMMARY OF THE INVENTION

It has now been surprisingly found that solubilized TCC may be incorporated into a gelled glycol deodorant stick composition to provide efficacious levels of TCC while maintaining translucency of the product. Solubilized triclocarban is formed by premixing the TCC with one or more solubilizers. The solubilized TCC may then be added to various deodorant stick compositions without concomitant production of opacity. Additionally, solubilized TCC provides a more efficacious delivery of the active bactericide to the skin.

The present invention is a solid deodorant composition minimally comprising solubilized triclocarban. In accordance with an exemplary embodiment of the present invention, the deodorant composition comprises triclocarban, at least one solubilizer, a structurant, and may also comprise volatile solvents, non-volatile solvents, emollients, waxes, antiperspirant actives, dyes, pigments, fragrances, etc. In a first exemplary embodiment, the translucent deodorant composition comprises solubilized TCC in a stearate-gelled glycol solvent deodorant stick.

### DETAILED DESCRIPTION OF THE INVENTION

The following description is of exemplary embodiments only and is not intended to limit the scope, applicability or configuration of the invention in any way. Rather, the following description provides a convenient illustration for implementing exemplary embodiments of the invention. Various changes to the described embodiments may be made in the function and relative amounts of components described without departing from the scope of the invention as set forth in the appended claims.

The present invention relates to a deodorant composition that incorporates triclocarban as the bactericide. As mentioned, triclocarban is insoluble in a hydrophilic, glycol matrix, and may be incorporated into a translucent deodorant stick at efficacious levels if it is previously solubilized. In a preferred embodiment, the triclocarban is intimately mixed with a solubilizer to form a solubilized TCC premix. That premix may then be added to some or all of the remaining ingredients of the deodorant stick (e.g., the structurant, the glycol solvents, silicones, etc.). Once cooled, the deodorant sticks show remarkable translucency and deodorant efficacy.

1. Solubilized 3,4,4′-trichlorocarbanilide (TCC)

The solid deodorant stick compositions of the present invention include “solubilized TCC”. As mentioned, triclocarban is notoriously insoluble in both aqueous, hydrophilic products and even in non-aqueous, hydrophobic products. To produce a translucent deodorant stick that comprises ICC as the active bactericidal ingredient would seem impossible given the poor solubility of TCC. However, it has been surprisingly discovered that TCC may be incorporated into a solid deodorant product at an antimicrobially effective level without adversely affecting the translucency of the stick, provided that the TCC is previously solubilized. In particular, the TCC is preferably solubilized into a “premix” that is subsequently added to some or a majority of the remaining ingredients in the deodorant product. Depending on the base chemistry (the “matrix”) of the deodorant chosen the preferred method for solubilizing the triclocarban to maintain a translucent final deodorant stick may differ. In particular, the nature of the solubilizer may be empirically chosen, or even mixtures of solubilizers may become necessary to maintain translucency of the stick containing TCC as the deodorant active.

Solubilized TCC has been previously described in the prior art. As far back as the introduction of this material by Bayer, better dispersion and antibacterial efficacy was known to be achievable by first dissolving the carbanilide in an organic solvent prior to mixing into a composition. For example, U.S. Pat. No. 4,547,307 (Hoppe, et al.) describes the use of solubilized triclocarban in a soap bar. The TCC was solubilized into the soap matrix using a small amount of alkyl phenol ethoxylate. Additionally, U.S. Pat. No. 5,922,768 (Gu) describes solubilization of ICC with a mixture of water soluble polyethylene glycol, having molecular weight of from about 500 to about 700, and a fragrance suitable for use in the final product. Most notably, Gu points out that polyethylene glycol alone fails to solubilize TCC, with precipitation invariably resulting. Thus the addition of a fragrance appears to provide some sort of co-solvent/solubilization effect. U.S. Pat. No. 5,977,049 (Briceno, et al.) describes solubilization of TCC into aqueous antibacterial compositions using a primary alcohol ethoxylate surfactant having general structure R—(OCH₂CH₂)ₙOH, wherein R is an alkyl group having 10-14 carbons, and wherein n is an average around 6 to 10. Briceno states that the required amount of surfactant solubilizer is about 6 to 15 times the weight of TCC ('049, Column 5, Lines 56-57).

For a solid deodorant stick as per the present invention, it has been found that solubilization of triclocarban (TCC) is best achieved by using singly or in combination; polyethylene glycols; polyethylene glycol mono alkyl ethers (i.e., alkoxy-polyethylene glycol); various polyalkoxylated glycerides; alkoxylated fatty acid amides; and, various quaternary salts. As mentioned, the solubilizer is first mixed with the TCC to form a premix that is then added to some or all of the remaining ingredients in the deodorant stick. With judicious choice of solubilizer(s), the amount required to maintain translucency in a stick containing TCC need only be about 2 to 5 times the weight % of the TCC.

The most preferred TCC solubilizers for use in the present invention include alkoxy-polyethylene glycol represented by the general formula; R—O—(CH₂CH₂O)ₙ—H, wherein n is the average number of oxyethylene repeating units (sometimes referred to as EO or PEG units, or “moles EO”), and is preferably from about 1 to about 25. Also preferred is that R is C₁-C₆ alkyl group. For example, R may be methyl, ethyl, n-propyl, iso-propyl, n-butyl, sec-butyl, iso-butyl, tert-butyl, pentyl, hexyl, cyclopropyl, cyclobutyl, cyclopropylmethylene, cyclopentyl, cyclobutylmethylene, cyclobutylethylene, cyclohexyl, cyclopropylpropylene, cyclobutylethylene, or cyclopentylmethylene. Most preferred for use herein, R is selected from the group consisting of methyl, ethyl, n-propyl, isopropyl, and cyclopropyl. In a preferred embodiment, the alkoxy-polyethylene glycol is methoxy-polyethylene glycol where R is methyl and n is from about 1 to about 25, or more preferably from about 2 to about 12, and most preferably from about 3 to about 10. Useful methoxy-polyethylene glycols include, for example, methoxy-diethylene glycol, methoxy-triethylene glycol, methoxy-tetraethylene glycol, methoxy-pentaethylene glycol, methoxy-hexaethylene glycol, methoxy-heptaethylene glycol, methoxy-octaethylene glycol, methoxy-nonaethylene glycol, methoxy-decaethylene glycol, methoxy-undecaethylene glycol, methoxy-dodecaethylene glycol, methoxy-tridecaethylene glycol, and methoxy-tetradecaethylene glycol. The ethylene glycol solubilizer may of course be used in the form of the single compound or as a mixture of two or more ranges of methoxy-(polyethylene) glycols. Most preferred, the alkoxy-polyethylene glycol is methoxy-polyethylene glycol 350 (Carbowax® MPEG-350, avg. MW of 350, n=about 7.2, from Dow), or methoxy-polyethylene glycol 550 (Carbowax® MPEG-550, avg. MW of 550, n=about 11.8, from Dow), or methoxy-polyethylene glycol 750 (Carbowax® MPEG-750, avg. MW of 750, n=about 16.3, from Dow), or combinations of these. By using one or more of the alkoxy-polyethylene glycols described herein, the solubilization of the triclocarban can be optimized, for example, with respect to overall solubility in the final deodorant stick and maintenance of translucency of the solidified product. As a solubilizer for the insoluble materials in a deodorant stick, an MPEG should be used at from about 0.1 to about 10 wt. % of the total composition. When MPEG (e.g. MPEG-350, MPEG-550, and/or MPEG-750 separate or in combination) is specifically used to solubilize TCC in a deodorant stick, the amount used should be about 5-times the amount of ICC. For example, where the TCC level is at about 0.3 wt. %, the amount of MPEG used to solubilize the TCC should be about 1.5 wt. %. Most preferred is to use MPEG-350 (avg. MW 350 methoxy polyethylene glycol) at about 0.1 to about 3 wt. % based on the total composition.

Preferred solubilizers also include polyethylene glycol (PEG) having molecular weights ranging from about 190 to about 9000 daltons. Such solubilizers include PEG-4 (MW 190-210), PEG-6 (MW 285-315), PEG-8 (MW 380-420), PEG-12 (MW 570-630), PEG-20 (MW 950-1050), PEG-32 (MW 1305-1595), PEG-75 (MW 3015-3685), PEG-90 (MW 3600-4400), PEG-100 (MW 4400-4800) and PEG-180 (MW 7000-9000), all available from Dow Chemical Co. under the Carbowax® brand. Of the PEGs, the most useful for solubilizing TCC in a deodorant stick has been PEG-12, (available from Dow as Carbowax® PEG 600, or Carbowax® PEG 600E). As a solubilizer for an insoluble antimicrobial in a deodorant stick, a PEG should be used at from about 0.1 to about 3 wt. % of the total composition. When PEG (e.g. PEG-12) is specifically used to solubilize TCC in a deodorant stick, the amount used should be about 5-times the amount of TCC. For example, where the TCC level is set at about 0.3 wt. %, the amount of PEG used to solubilize the TCC should be about 1.5 wt. %. Most preferred is to use PEG-12 or TPEG-900 (a Dow UCON® fluid) at about 0.1 to about 3 wt. % based on the total composition.

Solubilizers that may also find use in the deodorant sticks of the present invention include water soluble vegetable and animal-derived polyalkoxylated triglycerides; ethoxylated mono- and di-glycerides; polyethoxylated lanolins, and shea butter derivatives, and mixtures thereof. Suitable solubilizers of this class include polyethyleneglycol derivatives of glyceryl cocoate, glyceryl caproate, glyceryl caprylate, glyceryl tallowate, glyceryl palmate, glyceryl stearate, glyceryl laurate, glyceryl oleate, glyceryl ricinoleate, and glyceryl fatty esters derived from triglycerides, such as palm oil, almond oil, and corn oil, preferably glyceryl tallowate and glyceryl cocoate. Suitable solubilizers of this class are available from Croda Inc. (New York, USA) under their Crovol® brand and include Crovol® EP40 (PEG 20 evening primrose glyceride), Crovol® EP 70 (PEG 60 evening primrose glyceride) Crovol® A40 (PEG 20 almond glyceride), Crovol® A-70 (PEG 60 almond glyceride), Crovol® M-40 (PEG 20 maize glyceride), Crovol® M-70 (PEG 60 maize glyceride), Crovol® PK-40 (PEG 12 palm kernel glyceride), and Crovol® PK-70 (PEG 45 palm kernel glyceride), and under their Solan® brand, Solan® E, Solan® E50 and Solan® X polyethoxylated lanolins. Also preferred herein is PEG (6) capric/caprylic glyceride (Softigen® 767). Further suitable solubilizers of this type include, but are not limited to; PEG 80 glyceryl tallowate, PEG 28 glyceryl tallowate, PEG 200 glyceryl tallowate, PEG 30 glyceryl cocoate, and PEG 80 glyceryl cocoate. Of these materials, most preferred is to use Crovol® PK-70 (PEG 45 palm kernel glyceride) as the solubilizer for TCC, at a level of from about 0.1 wt. % to about 3 wt. % based on the total composition of the deodorant stick.

Additional solubilizers that may find use in the deodorant stick compositions of the present invention also include alkoxylated fatty acid amides. These solubilizers have general structure R—CO—NH—(CH₂CR″H—O)ₙ—R′, wherein, R is a C₈-C₂₄ fatty acid chain; n is between 1 and 9; R′=alkyl or hydroxyalkyl; and, R″ is hydrogen (for ethoxylated/EO series) or methyl (for propoxylated/PO series) or a mixture of hydrogen and methyl (for EO/PO series). One of the premier solubilizers in this family of chemicals is PPG-2 hydroxyethyl cocoamide (available as Promidium® CO from Mona Industries) and is preferably used at a level of from about 0.1 wt. % to about 3 wt. % based on the total composition of the deodorant stick.

Additional solubilizers that may find use in solubilizing the triclocarban in the compositions of the present invention include various quaternary ammonium salts. Such materials include, but are not limited to, alkylethyl morpholinium ethosulfate, cetylpyridinium chloride, isostearylethylimidonium ethosulfate, cocamidopropyl ethyldimonium ethosulfate, hydroxyethyl cetyldimonium chloride, quaternium-18, polyoxyethylene dihydroxypropyl linoleaminium chloride, bishydroxyethyl dihydroxypropyl stearaminium chloride, and cocodimonium hyroxypropyl hydrolyzed protein. Also useful are the phospholipids such as cocamidopropyl phosphatidyl PG-dimonium chloride, linoleamidopropyl phosphatidyl PG-dimonium chloride, and stearamidopropyl phosphatidyl PG-dimonium chloride. When a quaternary ammonium species is specifically used to solubilize TCC in a deodorant stick (singly or in combination with other solubilizers), the total amount used should be about 5-times the amount of TCC. For example, where the TCC level at about 0.3 wt. %, the amount of quaternary used to solubilize the TCC should be about 1.5 wt. %. Most preferred is to use a combination of two solubilizers comprising isostearyl ethylimidonium ethosulfate and methoxy polyethylene glycol (MPEG-350 with avg. MW 350) at a total of from about 0.1 to about 3 wt. % based on the total composition.

2. Structurant

The anhydrous antiperspirant stick compositions of the present invention comprise a suitable concentration of a solid structurant to help provide the compositions with the desired product hardness, or to otherwise help suspend any dispersed solids or liquids within the composition. The term “solid structurant” as used herein refers to any material known or otherwise effective in providing suspending, gelling, viscosifying, solidifying and/or thickening to the composition or to otherwise provide structure to the final product form. These solid structurants include gelling agents, polymeric, non-polymeric or inorganic thickening agents, and/or viscosifiers. Such materials will typically be solids under ambient conditions and include organic solids (waxes, fats, fatty acid salts, hydrogenated oils, and the like), crystalline or other gellants, cellulosic materials, various sorbitols, inorganic particulates such as clays, talc, silica or fumed/treated silicas, and combinations thereof. The concentration and type of solid structurant selected for use in the present deodorant compositions will vary depending upon the desired product hardness, rheology, and/or other related product characteristics, and it is common to use mixtures of structurants. For most structurants suitable for use herein, the total structurant concentration ranges from about 1% to about 40% wt. % of the total stick composition.

Non-limiting examples of suitable structurants include cellulosic thickeners such as hydroxy propyl cellulose and hydroxy ethyl cellulose, sodium stearate, stearyl alcohol, hydroxystearic acid, dibutyl lauroyl glutamide, alkyl silicone waxes, quaternium-18 bentonite, quaternium-18 hectorite, propylene carbonate, hydrogenated castor or jojoba oil (e.g., Castorwax MP80, Castor Wax, etc.); natural animal or plant oils/waxes and semi-synthetic oils/waxes such as avocado oil, linseed oil, almond oil, Ibota wax, perilla oil, olive oil, cacao butter, kapok wax, kaya oil, carnauba wax, glycyrrhiza oil, candelilla wax, beef tallow, neat's-foot oil, beef bone fat, hydrogenated beef tallow, apricot kernel oil, spermaceti wax, hydrogenated oils such as wheat germ oil, sesame oil, rice germ oil, rice bran oil, sugar cane wax, sasanqua oil, safflower oil, shear butter, Chinese tung oil, cinnamon oil, jojoba wax, shellac wax, turtle oil, soybean oil, tea seed oil, camellia oil, evening primrose oil, corn oil, lard, rapeseed oil, Japanese tung oil, rice bran oil, germ oil, horse fat, persic oil, palm oil, palm kernel oil, castor oil, castor oil fatty acid methylester, sunflower oil, grape oil, bayberry wax, jojoba oil, macadamia nut oil, beeswax, mink oil, cottonseed oil, cotton wax, Japanese wax, Japanese wax kernel oil, montan wax, coconut oil, hydrogenated coconut oil, tri-coconut oil fatty acid glyceride, mutton tallow, peanut oil, lanolin, liquid lanolin, hydrogenated lanolin, lanolin alcohol, hard lanolin, lanolin acetate, isopropyl lanolate, hexyl laurate, poly(oxyethylene) lanolin alcohol ether, poly(oxyethylene) lanolin alcohol acetate, polyethylene glycol lanolate, poly(oxyethylene) hydrogenated lanolin alcohol ether, egg yolk oil, the synthetic waxes such as Fisher-Tropsch waxes and microcrystalline wax; polyethylene with molecular weight of 200 to 1000 daltons, polyethylene-vinyl acetate copolymers, substituted and unsubstituted dibenzylidene alditols (e.g. dibenzylidene sorbitol); silica, fumed silica (hydrophobic or hydrophilic treated, e.g. Aerosil 300, and/or R972 from Evonik-Degussa), talc, and solid triglycerides, and combinations thereof.

The most preferred waxy structurants include fatty alcohols such as stearyl alcohol and myristyl alcohol, the hydrogenated oils such as hydrogenated castor oil, Jojoba oil and lanolin, paraffin wax, beeswax, carnauba wax, candelilla wax, spermaceti wax, ozokerite (ozocerite) wax, ceresin wax, and montan wax, and mixtures thereof. The most preferred inorganic structurants include silica, such as the Aerosil silicas, fumed silica (most notably hydrophobic treated fumed silica) and talc.

Most preferred structurants for use herein include hydroxy propyl cellulose, hydroxy ethyl cellulose, dibenzylidene sorbitol, sodium stearate, stearyl alcohol, cetyl alcohol, paraffin waxes, hydroxystearic acid and sodium hydroxystearate, dibutyl lauroyl glutamide, hydrogenated fats (e.g. Castor wax); silica; talc; and, fumed silica (e.g. Aerosil 300, Aerosil R972), and mixtures thereof; with the structurant combination totaling from about 1% to about 45 wt. %. Most preferred is to use sodium stearate at from about 1-10 wt. %, and/or various combinations of hydrogenated Castor wax, silica, fumed silica and talc, at a total of from about 20 wt. % to about 45 wt. %.

3. Silicones

The deodorant compositions of the present invention may also include one or more volatile silicones. These materials include the cyclic polydimethylsiloxanes, also known as cyclomethicones, which preferably have from about 3 to about 6 silicon atoms, and the linear polydimethylsiloxanes, also known as dimethicones, which preferably have from about 2 to about 9 silicon atoms. The cyclomethicones normally exhibit viscosities below 10⁻⁵ m²/sec (10 centistokes) and above 10⁻⁷ m²/sec (0.1 centistokes), and the dimethicones normally exhibit a viscosity of below 5×10^{-6 m}²/sec (5 centistokes). Cyclomethicones may include the silicone fluids DC® 244, DC® 245, DC® 246, DC® 344, and DC® 345, all of which are available from Dow Corning Corporation, Silicone 7207® and Silicone 7158® from Union Carbide Corporation; and SF1202® from General Electric. In at least one preferred embodiment of the deodorant stick of the present invention, the volatile silicone fluid comprises cyclomethicone (particularly decamethylpentasiloxane). In the formulations of this invention, the volatile silicone fluid may be present in the deodorant stick composition in an amount of from 20 to 70 wt. % and most preferably at about 40 to 60 wt. %. In at least one embodiment of particular interest, the volatile silicone is present in the stick composition at a level of about 50 wt. %.

The deodorant compositions may also include one or more non-volatile silicones such as polyalkylsiloxanes, polyalkylaryl siloxanes, and polyether siloxanes with viscosities of about 5 to about 100,000 centistokes at 25° C., polymethylphenylsiloxanes with viscosities of about 15 to about 65 centistokes, and polyoxyalkylene ether dimethylsiloxane copolymers with viscosities of about 1200 to about 1500 centistokes. These materials also function as emollients as described below. Non-volatile silicones may be incorporated in the deodorant sticks of the present invention at from about 0.1 wt. % to about 15 wt. %.

4. Emollient

An emollient (e.g., a non-volatile emollient) may be incorporated in the deodorant stick composition of the present invention to impart desirable feel to the stick itself and to render emollient effects to the skin to which the product is applied. Suitable non-volatile emollients include silicone and non-silicone materials. Such silicone materials include polyalkyl siloxanes, polyalkyaryl siloxanes, and polyether siloxane copolymers. Non-silicone materials may include fatty acids, fatty alcohol esters (e.g. “ester oils”), and water insoluble ethers and alcohols. Emollients typically used in personal care compositions are described in Cosmetic, Science and Technology, Vol, 1, 27-104 (1972, Balsam and Sagarin editors), incorporated herein by reference in its entirety. Emollients may be incorporated in the deodorant composition from about 3% to about 27% by weight of the total composition. Particular examples of emollients include PEG-14 butyl ether, which is most useful when incorporated at about 4-15% wt. %; phenyl trimethicone at about 2-10 wt. %; C₁₂-C₁₅ alkyl benzoate at about 4-15 wt. %; and/or dimethicone in an amount of about 2-5 wt. %. Of course, combinations of these emollients may find use in the deodorant sticks of the present invention.

Also added as emollients are polyethylene and/or polypropylene glycol ethers of C₄₋₂₀ alcohols, including such materials as PPG-10 Butanediol, PPG-14 Butyl Ether, PPG-5-Buteth-7, PPG-3-Isostearth-9, PPG-3-Myreth-3, Oleth-10, and Steareth-20. Polyethylene and/or polypropylene glycol esters of fatty acids include PEG-8 Distearate, PEG-10 Dioleate, and PPG-26 Oleate.

Ester oils are also preferred as emollients in the present invention. Examples of the ester oils include diisobutyl adipate, 2-hexyldecyl adipate, di-2-heptylundecyladipate, N-alkyl glycol monoisostearate, isocetyl isostearate, trimethylolpropane triisostearate, ethylene glycol di-2-ethylhexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, isononyl isononanate, neopentyl glycol dicaprirate, triethyl citrate, 2-ethylhexyl succinate, amyl acetate, ethyl acetate, butyl acetate, isocetyl stearate, butyl stearate, diisopropyl sebacinate, di-2-ethylhexyl sebacinate, cetyl lactate, myristyl lactate, isopropyl palmitate, 2-ethylhexyl palmitate, 2-hexyldecyl palmitate, 2-heptylundecyl palmitate, cholesteryl 12-hydroxystearate, dipentaerythritol fatty acid esters, isopropyl myristate, octyldodecyl myristate, 2-hexyldecyl myristate, myristyl myristate, hexyldecyl dimethyloctanoate, ethyl laurate, hexyl laurate, 2-octyldodecyl N-lauroyl-L-glutamate, and diisostearyl malate; and glyceride oils, e.g., acetoglyceryl, glycerol triisooctanoate, glyceryl triisostearate, glyceryl triisopalmitate, glyceryl monostearate, glyceryl di-2-heptylundecanoate, glyceryl trimyristate, and diglyceryl myristyl isostearate. When used in a deodorant stick composition, one or more ester oils may be used at from about 0.1 wt. % to about 10 wt. %. More preferred is to use myristyl myristate at from about 0.5 wt. % to about 3.0 wt. %. Most preferred is to use a mixture of PPG-14 butyl ether at from about 4 wt. % to about 15 wt. % and myristyl myristate at from about 0.5 wt. % to about 3.0 wt. % in the deodorant stick of the present invention.

5. Solvent Component

The deodorant stick compositions of the present invention preferably include at least one solvent. Most preferred are the hydric solvents such as water, alcohols, glycols, and polyols. These substances may provide benefits such as moisture retention, rheology modification, or even bacteriostatic/bactericidal action. Preferred for use herein include materials such as water, glycerin, sorbitol, propylene glycol, dipropylene glycol, 1,3-butylene glycol, pentylene glycol, hexylene glycol, glucose, xylitol, malitol, polyethylene glycol or polypropylene glycol (typically having molecular weights of about 500 to 6000, such as PEG-10, PEG-40, PEG-150 and PPG-20), pyrrolidone carboxylate, polyoxyethylene glycoside, and polyoxypropylene methylglycoside.

Solvents for use herein may also include volatile and/or non-volatile hydrocarbons. Non-volatile paraffinic hydrocarbons include mineral oils and branched-chain hydrocarbons with about 16 to 68, preferably about 20 to 40, carbon atoms. An example is hydrogenated poly-(isobutene) with about 24 carbon atoms.

For the deodorant stick compositions of the present invention, the preferred solvent level is from about 0.1 wt. % to about 80 wt. %, depending on the matrix for the stick and the nature of the solvents. For example, a preferred embodiment may contain from about 25 wt. % to about 80 wt. % of a single glycol or a mixture of glycols. Most preferred is to use a combination of propylene glycol and dipropylene glycol at a total of from about 60 wt. % to about 80 wt. %, and most preferably at a ratio close to 1:1 at a total of about 70-75 wt. %. Also preferred is to incorporate water as the only hydric solvent, or as a mixture of solvents, at a level of from about 0.1 wt. % to about 20 wt. %.

6. Optional Additional Deodorant Active

The deodorant compositions of the present invention may further comprise additional antimicrobial actives besides the 3,4,4′-trichlorocarbanilide to further help prevent or eliminate malodors resulting from odor causing microbes. Indeed, some of these actives are known to have synergistic effects with triclocarban. The concentration of the optional additional deodorant active may range from about 0.001% to about 20%, and preferably from about 0.1% to about 1%, by weight of the composition. Suitable optional additional deodorant actives may include quaternary ammonium salts (e.g. cetyltrimethylammonium chloride or bromide; tricetylmethylammonium chloride or bromide), protonated trialkylamines (i.e., trialkylammonium salts), cetylpyridinium salts, benzethonium chloride, diisobutyl phenoxy ethoxyethyl dimethylbenzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, diglycerol monocaprate, diglycerol monolaurate, glycerol monolaurate, polyhexamethylene biguanide salts, sodium aluminum chlorohydroxy lactate, triethyl citrate, 2,2′-dihydroxy-3,5,6,3′,5′,6′-hexachlorodiphenylmethans (hexachlorophene), 3-trifluoromethyl-4,4′-dichloro-N,N′ diphenyl urea (Irgasan CF₃), 2,4,4′-trichloro-2′-hydroxy diphenyl ether (triclosan), diaminoalkyl amides such as L-lysine hexadecylamide, miscellaneous citrates and salicylates, zinc pyrithione, zinc phenolsulfate, 3,7,11-trimethyldodeca-2,6,10-trienol(farnesol), usnic acid salts, zinc phenolsulfonate, b-chloro-D-alanine, D-cycloserine, aminooxyacetic acid, cyclodextrin, and sodium bicarbonate, and combinations thereof.

7. Optional Antiperspirant Active

The deodorant compositions of the present invention may comprise antiperspirant active. The concentration of antiperspirant active should be sufficient to provide the desired perspiration wetness control from the deodorant composition (wax-stick or soap-stick) composition selected. The deodorant compositions of the present invention may comprise an antiperspirant active at concentrations ranging from about 0.1% to about 30% by weight of the composition. The antiperspirant active can be solubilized or incorporated as dispersed solid particulates, for example with average particle size of from about 1 μm to about 40 μm.

The antiperspirant active that may be used in the deodorant compositions of the present invention include astringent metallic salts, especially inorganic and organic salts of aluminum, zirconium and zinc, as well as mixtures thereof. Particularly preferred are aluminum-containing and/or zirconium-containing salts or materials, such as aluminum halides, aluminum chlorohydrate, aluminum hydroxyhalides, zirconyl oxyhalides, zirconyl hydroxyhalides, and mixtures thereof.

Preferred aluminum salts for use in the anhydrous antiperspirant embodiments of the present invention include those having the generalized formula Al₂(OH)ₐCl_{b}x(H₂O), wherein a is from about 2 to about 5; a and b total to about 6; x is from about 1 to about 6; and wherein a, b, and x may have non-integer values.

Preferred zirconium salts for use in the deodorant compositions of the present invention include those having generalized formula ZrO(OH)₂₋ₐClₐx(H₂O), wherein a is from about 1.5 to about 1.87; x is from about 1 to about 7; and wherein a and x may both have non-integer values. Particularly preferred zirconium salts are those complexes which additionally contain aluminum and glycine, commonly known as ZAG complexes. These ZAG complexes contain aluminum chlorohydroxide and zirconyl hydroxy chloride conforming to the above described formulas.

Optional antiperspirant actives that may find use in the compositions of the present invention include aluminum chlorohydrate, aluminum dichlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum zirconium trichlorohydrate, aluminum zirconium tetrachlorohydrate, aluminum zirconium pentatchlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, aluminum chloride, aluminum sulfate buffered, and combinations thereof.

8. Fragrance Component

In addition to the solubilized TCC, the structurants, solvents, emollients, silicones and the like, the deodorant stick compositions of the present invention preferably include a fragrance component. The fragrance component is separate and distinct from the deodorant active in the invention. That is, the invention necessarily includes triclocarban (TCC) as the active deodorant agent, and may include additional deodorants that are necessarily antimicrobial, whereas the fragrance is optionally added to the present invention for greater sensorial appeal and possible odor masking/complexing.

Such fragrances for use herein may include a broader class of natural products comprising natural oils extracted from plants and trees and their fruits, nuts and seeds, (for example by steam or liquid extraction of ground-up plant/tree material), natural products that may be purified by distillation, (i.e., purified single organic molecules or close boiling point “cuts” of organic materials such as terpenes and the like), and synthetic organic materials that are the synthetic versions of natural materials (e.g., either identical to the natural material or perhaps the optical isomer, or the racemic mixture). An example of the latter is d,l-limonene that is synthetically prepared and is a good and eco-friendly substitute for natural orange oil (mostly d-limonene) when crop yields are expensive due to citrus crop freezes. It is understood that “fragrances” usually incorporate a wide range of pure organic materials either natural or synthetic, mixtures of these previously purified individual materials or distillate cuts of materials, and complex natural mixtures directly extracted from plant/tree materials through infusion, steam extraction, etc., and in fact many natural extracts, oils, essences, infusions and such are very fragrant materials. Fragrances for use in personal care compositions are available from the fragrance supply houses such as International Flavors & Fragrances (IFF), Symrise, Givaudan, Firmenich, Robertet, H&R, amongst others, and may be added to the deodorant stick to provide both odor masking, complexing of odorous materials through fragrance accords, or general “perfuming” for the product itself and to impart substantive fragrance to the skin.

Some of the naturally derived materials that may be of use in the present compositions include, but are not limited to, musk, civet, ambergis, castoreum and similar animal derived oils; abies oil, ajowan oil, almond oil, ambrette seed absolute, angelic root oil, anise oil, basil oil, bay oil, benzoin resinoid, bergamot oil, birch oil, boil de rose oil, broom abs., cajeput oil, cananga oil, capsicum oil, caraway oil, cardamon oil, carrot seed oil, cassia oil, cedar leaf oil, cedar wood oil, celery seed oil, cinnamon bark oil, citronella oil, clary sage oil, clove oil, cognac oil, coriander oil, cubeb oil, cumin oil, camphor oil, dill oil, elemi gum, estragon oil, eucalyptol nat., eucalyptus oil, fennel sweet oil, galbanum res., garlic oil, geranium oil, ginger oil, grapefruit oil, hop oil, hyacinth abs., jasmin abs., juniper berry oil, labdanum res., lavender oil, laurel leaf oil, lavender oil, lemon oil, lemongrass oil, lime oil, lovage oil, mace oil, mandarin oil, mimosa abs., myrrh abs., mustard oil, narcissus abs., neroli bigarade oil, nutmeg oil, oakmoss abs., olibanum res., onion oil, opoponax res., orange oil, orange flower oil, origanum, orris concrete, pepper oil, peppermint oil, peru balsam, petitgrain oil, pine needle oil, rose abs., rose oil, rosemary oil, safe officinalis oil, sandalwood oil, sage oil, spearmint oil, styrax oil, thyme oil, tolu balsam, tonka beans abs., tuberose abs., turpentine oil, vanilla beans abs., vetiver oil, violet leaf abs., ylang ylang oil and similar vegetable oils, etc.

Synthetic essences include but are not limited to pinene, limonene and like hydrocarbons; 3,3,5-trimethylcyclohexanol, linalool, geraniol, nerol, citronellol, menthol, borneol, borneyl methoxy cyclohexanol, benzyl alcohol, anise alcohol, cinnamyl alcohol, β-phenyl ethyl alcohol, cis-3-hexenol, terpineol and like alcohols; anethole, musk xylol, isoeugenol, methyl eugenol and like phenols; α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, citral, citronellal, hydroxycitronellal, benzaldehyde, methyl nonyl acetaldehyde, cinnamic aldehyde, dodecanol, α-hyxylcinnamic aldehyde, undecenal, heliotropin, vanillin, ethyl vanillin and like aldehydes; methyl amyl ketone, methyl β-naphthyl ketone, methyl nonyl ketone, musk ketone, diacetyl, acetyl propionyl, acetyl butyryl, carvone, menthone, camphor, acetophenone, p-methyl acetophenone, ionone, methyl ionone and like ketones; amyl butyrolactone, diphenyl oxide, methyl phenyl glycidate, gamma.-nonyl lactone, coumarin, cineole, ethyl methyl phenyl glicydate and like lactones or oxides; methyl formate, isopropyl formate, linalyl formate, ethyl acetate, octyl acetate, methyl acetate, benzyl acetate, cinnamyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, methyl salicylate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, nopyl acetate, β-phenylethyl acetate, trichloromethylphenyl carbinyl acetate, terpinyl acetate, vetiveryl acetate and the like.

Encapsulated fragrances are well known in the art, and may find use in the deodorant stick of the present invention to give the product a longer-lasting fragrance impression (better retention of the fragrance in storage) along with extended fragrance release on the skin (through mechanical or moisture/sweat rupture of the fragrance caps). Encapsulation of fragrance has been described in many prior art references, including but not limited to; U.S. Pat. No. 7,338,928 to Lau et al.; U.S. Pat. No. 7,294,612 to Popplewell et al.; U.S. Pat. No. 7,196,049 to Brain et al.; U.S. Pat. No. 7,125,835 to Bennett et al.; U.S. Pat. No. 7,122,512 to Brain et al.; U.S. Pat. No. 7,119,057 to Popplewell et al.; U.S. Pat. No. 6,147,046 to Shefer et al.; U.S. Pat. No. 6,142,398 to Shefer et al.; U.S. Pat. No. 4,446,032 to Munteanu et al.; and, U.S. Pat. No. 4,464,271 to Munteanu, each of which is incorporated herein by reference. Fragrance encapsulation has been optimized in the trade and is readily available through various suppliers, most notably LIPO Technologies, Inc., Vandalia, Ohio, and Alco Chemical, Chattanooga, Tenn., (e.g. using Alcocap® natural polymers for encapsulation): Encapsulation is described thoroughly in “Microencapsulation: Methods and Industrial Applications”, Benita (Ed.), Marcel Dekker, Inc., New York, 1996. Fragrance microcapsules obtained from LIPO, Alco, or the fragrance houses, or as obtained through any of these published methods may be incorporated in the deodorant sticks herein at from about 0.001% to about 5% by weight of the total composition.

9. Optional Adjuvant

The deodorant sticks of the present invention may also include, dyes, pigments, UV filters/blockers, depilatory agents, preservatives, skin pigment modifiers (e.g. instant tan technology), vitamins, conditioners, anti-wrinkle agents, and the like. Dyes are optional ingredients within the compositions of the present invention since color may be more effectively incorporated within the dispensing package itself, unless the package is clear plastic and the composition itself preferably colored. Dyes may comprise pigments, or other colorants, chosen so that they are compatible with the other ingredients in the deodorant composition, compatible with the manufacturing process, and not staining to skin or clothing. For example, a preferred colorant for use in the present invention is at from about 0.0001% to about 0.1% by weight of the total composition. Non-limiting examples of dyes include Blue 1 (CI 420980), Yellow 8 (CI 43350), Liquitint® Green FS (from Milliken), C.I. Pigment Green #7, C.I. Reactive Green #12, F D & C Green #3, C.I. Acid Blue #80, C.I. Acid Yellow #17, Liquitint® Red MX, F D & C Yellow #5, Liquitint® Violet LS, Fast Turquise GLL, Liquitint® Blue MC, and Liquitint® Blue HP, and mixtures thereof.

Examples of suitable UV filters include 4-aminobenzoic acid; 3-(4′-trimethylammonium)benzylideneboran-2-one methylsulfate; 3,3,5-trimethylcyclohexyl salicylate; 2-hydroxy-4-methoxybenzophenone; 2-phenyl benzimidazole-5-sulfonic acid and its potassium, sodium, and triethanolamine salts; 3,3′-(1,4-phenylenedimethine)bis-(7,7-dimethyl-2-oxobicyclo[2.2.1]heptane-1-methanesulfonic acid and its salts; 1-(4-tert-butylphenyl)-3-(4-methoxyphenyl)propane-1,3-dione, 3-(4′-sulfo)benzylidene-bornan-2-one and its salts; 2-ethylhexyl 2-cyano-3,3-diphenylacrylate; polymer of N-[2(and 4)-(2-oxoborn-3-ylidenemethyl)benzyl]acrylamide; 2-ethylhexyl 4-methoxycinnamate; ethoxylated ethyl 4-aminobenzoate; isoamyl 4-methoxycinnamate; 2,4,6-tris[p-(2-ethylhexyloxycarbonyl)anilino]-1,3,5-triazine; 2-(2H-benzotriazol-2-yl)-4-methyl-6-(2-methyl-3-(1,3,3,3-tetramethyl-1-(trimethylsilyloxy)silyloxy)disiloxanyl)propyl)phenol; 4,4′-[(6-[4-((1,1-dimethylethyl)aminocarbonyl)-phenylamino]-1,3,5-triazin-2,4-yl)diimino]bis(benzacid 2-ethylhexyl ester); 3-(4′-methylbenzylidene) D,L-camphor; 3-benzylidene camphor; 2-ethylhexylsalicylate; 2-ethylhexyl-4-dimethylaminobenzoate; hydroxy-4-methoxybenzophenone phenone-5-sulfonic acid (sulisobenzone) and the sodium salt; and, 4-isopropylbenzyl salicylate, and mixtures thereof.

Example Deodorant Stick Compositions and Methods of Manufacture

With the necessary and optional ingredients thus described, exemplary embodiments of the deodorant composition are shown in the table:

| | Exam- | Exam- | Exam- |
|---|---|---|---|
| Ingredients (wt. % active/theoretical) | ple 1 | ple 2 | ple 3 |
| 3,4,4′-Trichlorocarbanilide (Triclocarban) | 0.3 | 0.1 | 0.3 |
| Cyclopentasiloxane | 50.0 | — | — |
| Stearyl alcohol | 24.0 | — | — |
| PPG-14 butyl ether | 9.8 | — | — |
| Hydrogenated Castor Wax | 2.8 | — | — |
| Myristyl myristate | 1.9 | — | — |
| Silica dimethyl silylate (fumed, | 1.4 | — | — |
| hydrophobic silica) | | | |
| Silica | 0.3 | — | — |
| Talc | 9.8 | — | — |
| Propylene glycol | — | 35.5 | 36.5 |
| Dipropylene glycol | — | 35.5 | 36.2 |
| Sodium Stearate | — | 8.5 | 8.5 |
| PEG-6 methyl ether (methoxy | — | — | 1.5 |
| polyethylene glycol) | | | |
| Water, dyes, fragrance, other adjuvant | q.s. | q.s. | q.s. |

Three examples are shown in the table above. Example 1 is a “wax-stick” and does not comprise solubilized TCC. Example 2 is a “soap-stick” with a reduced level of TCC that may be formulated without the use of solubilizers. Example 3 is an embodiment of the present invention wherein an efficacious amount of TCC (about 0.3 wt. %) is solubilized into the soap-stick base using about 1.5 wt. % of MPEG-350 solubilizer. Example 1 is quite opaque. Example 3 is quite translucent, whereas Example 2 is somewhat in between in appearance.

The wax stick (Example 1) was made by charging the mixing vessel with a first portion of cyclopentasiloxane (about 90% of the total cyclopentasiloxane required for the batch). Agitation was started and the silica, fumed silica and triclocarban were added sequentially with agitation. Then the talc was added, with mixing continued until these inorganic substances were wetted and dispersed. In a separate vessel, the castor wax, stearyl alcohol, PPG-14 butyl ether and myristyl myristate were added sequentially. Heat was introduced to this vessel to melt the waxy materials within, and agitation was slowly started as the mix became molten. The temperature of the molten mixture was kept below about 85° C. To this agitated and molten mixture was added the premix of triclocarban, siloxane and the inorganic substances. Lastly, the second portion of cyclopentasiloxane (the remaining 10% required) was added and mixing continued until homogeneous. The heat was terminated and the mixture cooled to about 53° C. before pouring into separate deodorant stick dispensers.

The soap-stick Example 2 without solubilized TCC was made by first adding propylene glycol and dipropylene glycol to the vessel. Agitation was then started and the TCC was added and thoroughly dispersed. The remaining ingredients were then added, with continuous agitation maintained and heating applied to achieve a batch temperature of about 85° C. The heat was then terminated and the mixture cooled to about 65° C. before pouring into separate deodorant stick dispensers.

For the soap-stick Example 3 comprising solubilized TCC at the higher and efficacious level of 0.3 wt. %, the propylene glycol and dipropylene glycol were added to a vessel and agitation was started. To the stirred glycol blend was added a premix previously prepared in a separate vessel and comprising triclocarban and PEG-6 methyl ether (MPEG-350 methoxy polyethylene glycol). The resulting mixture was stirred until well dispersed and clear. The remaining ingredients were added while the heat was ramped up to about 85° C. The heat was removed and the mixture cooled to about 65° C. before pouring into separate deodorant stick dispensers.

To demonstrate the deodorant characteristics of the various personal care deodorant compositions of the present invention, Example 1 (opaque solid deodorant stick with 0.3% TCC) was compared to Example 3 (translucent solid deodorant stick with 0.3% solubilized TCC). The results appear in the following table.

| | Example 1 | Example 3 |
|---|---|---|
| | (opaque wax-stick) | (translucent soap-stick) |
| Mal- | Both the active solid stick and | Both the active translucent stick |
| odor | placebo solid stick significantly | and placebo solid stick |
| | decreased malodor at 10 and 24 | significantly decreased malodor |
| | hours post treatment. | at 10 and 24 hours post |
| | There was a trend at 10 hours | treatment. |
| | for significantly less malodor | There was no difference |
| | with active treatment, but there | between active translucent stick |
| | was no difference between | and placebo at 10 or 24 hours. |
| | active and placebo at 24 hours. | |
| Bac- | At 10 hours there was no | At 10 hours there was no |
| teria | difference between active solid | difference between active |
| | stick and placebo for either | translucent stick and placebo for |
| | diphtheroids or total aerobic | either diphtheroids or total |
| | bacteria. | bacteria. |
| | At 24 hours there was a trend | At 24 hours there was |
| | (p = 0.0772) to significantly | significantly fewer diphtheroids |
| | fewer diphtheroids with active | (p = 0.0129) and total aerobic |
| | treatment and a significant | bacteria (p < .0001) with active |
| | difference (p = 0.0049) in total | translucent stick. |
| | aerobic bacteria favoring the | |
| | active solid stick. | |

We have thus described translucent solid deodorant stick compositions comprising solubilized TCC. The solubilization of the TCC as a premix is required for the production of translucent deodorant products that provide bactericidal efficacy on gram positive bacteria.

We claim:

1. A personal care deodorant composition comprising:
a. from about 0.01% to about 1.0% by weight of 3,3,4′-trichlorocarbanilide;
b. from about 0.1% to about 3% by weight of a solubilizer;
c. from about 1% to about 45% by weight of a structurant; and
d. from about 0.1% to about 80% by weight of a hydric solvent,
wherein the ratio of said solubilizer to said 3,3,4′-trichlorocarbanilide is from about 1 to about 6.

2. The personal care deodorant composition of claim 1, wherein said solubilizer is chosen from the group consisting of polyethylene glycols, polyethylene glycol mono alkyl ethers, polyalkoxylated glycerides, alkoxylated fatty acid amides, alkylethyl morpholinium ethosulfate, cetylpyridinium chloride, isostearylethylimidonium ethosulfate, cocamidopropyl ethyldimonium ethosulfate, hydroxyethyl cetyldimonium chloride, quaternium-18, polyoxyethylene dihydroxypropyl linoleaminium chloride, bishydroxyethyl dihydroxypropyl stearaminium chloride, and cocodimonium hyroxypropyl hydrolyzed protein, and mixtures thereof.

3. The personal care deodorant composition of claim 1, wherein the structurant is chosen from the group consisting of fatty alcohol waxes, fatty acid salts, dibenzylidine alditol, hydrogenated oils, clay, talc, silica, and fumed silica, and mixtures thereof.

4. The personal care deodorant composition of claim 1, wherein the hydric solvent is chosen from the group consisting of water, glycerin, propylene glycol, dipropylene glycol, 1,3-butylene glycol, pentylene glycol, hexylene glycol, polyethylene glycol of molecular weight from about 500 to about 6000 daltons, and polypropylene glycol of molecular weight from about 500 to about 6000 daltons, and mixtures thereof.

5. The personal care deodorant composition of claim 2, wherein said solubilizer is 0.1% to about 3.0% by weight methoxy polyethylene glycol having molecular weight from about 350 to about 750 daltons.

6. The personal care deodorant composition of claim 4, wherein said hydric solvent is chosen from the group consisting of water, propylene glycol, and dipropylene glycol, and mixtures thereof.

7. A translucent solid deodorant composition comprising:
a. from about 0.1% to about 0.5% by weight 3,4,4′-trichlorocarbanilide;
b. from about 0.1% to about 3% by weight methoxy polyethylene glycol having molecular weight from about 350 to about 700 dalton;
c. from about 1% to about 10% of a fatty acid salt; and,
d. from about 60% to about 80% by weight of a mixture of propylene glycol and dipropylene glycol,
wherein the ratio of said solubilizer to said 3,3,4′-trichlorocarbanilide is from about 1 to about 6.

8. The solid deodorant composition of claim 7, wherein said fatty acid salt is sodium stearate.

9. The solid deodorant composition of claim 7, wherein said methoxy polyethylene glycol is PEG-6 methyl ether with molecular weight around 350 daltons.

10. A method of producing the solid deodorant composition of claim 7, said method comprising the steps of:
a. mixing together said propylene glycol and dipropylene glycol and heating the resulting mixture to about 85° C.;
b. forming a premix of said 3,3,4′-trichlorocarbanilide and said methoxy polyethylene glycol;
c. adding said 3,3,4′-trichlorocarbanide/methoxy polyethylene glycol premix to said heated mixture of glycols;
d. adding said fatty acid salt to said heated mixture;
e. adding any optional adjuvant; and,
f. allowing said heated mixture to cool to about 65° C. and casting said mixture into separate personal care dispensing containers.
